(19) United States
(12) Patent Application Publication
(10) Pub. No.: US 2021/0113323 A1
(43) Pub. Date: Apr. 22, 2021

(54) MUCOSAL EXCLUSION ANASTOMOSIS DEVICE AND METHODS

(71) Applicant: University of Chicago, Chicago, IL (US)

(72) Inventor: Herbert M. Hedberg, Chicago, IL (US)

(21) Appl. No.: 17/077,509

(22) Filed: Oct. 22, 2020

Related U.S. Application Data
(60) Provisional application No. 62/924,305, filed on Oct. 22, 2019.

(51) Int. Cl. A61F 2/06; A61L 27/22; A61L 27/20

(52) U.S. Cl. CPC A61F 2/064; A61L 27/227; C08L 5/08; A61F 2220/005; A61L 2430/22; A61L 27/20

(57) ABSTRACT

A coupling device for constructing bowel anastomoses between a first tubular segment and a second tubular segment is provided. The coupling device includes a wall having a first end and an opposing second end. The wall defines a bore. The first end is sized and shaped for insertion into the first tubular segment. The second end is sized and shaped for insertion into the second tubular segment. The wall includes a mucoadhesive polymer configured to adhere to the first tubular segment and the second tubular segment. After the tubular segments are positioned upon the coupling, the tubular segments may be joined together to stabilize the connection.

### CROSS-REFERENCE TO RELATED APPLICATIONS

This application claims priority of U.S. Provisional Application No. 62/924,305, filed Oct. 22, 2019, which is hereby incorporated by reference in its entirety.

### FIELD OF THE DISCLOSURE

The field of the present disclosure relates generally to surgical anastomoses and, more particularly, to devices, systems, and methods for constructing adhesive anastomoses between two opposing tubular segments of intestine.

### BACKGROUND

An anastomosis is a surgical connection between two tubular segments, e.g., two opposing cut bowel segments. Conventionally, a bowel anastomosis is fashioned using sutures and/or staples to invert and join the cut bowel edges. Sutures and stapes create a network of microscopic channels in the tissue, and in combination with an inverted cut bowel edges, exposes a large surface area of collagen connective tissue at the anastomotic wound.

Healing of anastomotic wounds may be impaired by the adherence of gut bacteria to exposed collagen. E. faecalis, a ubiquitous commensal gut flora, is capable of adhering to collagen fibers exposed in the anastomotic wound on the inverted edges. Driven by a phosphate-seeking mechanism, collagen adherence may induce phenotypic transformation to begin collagenase production and tissue invasion. Bacteria can also modulate normal collagen remodeling in the healing wound by upregulating matrix metalloproteinases (MMPs), which accelerate collagen degradation. E. faecalis collagenase production is associated with increased MMP activity and anastomotic leak. Leaks are devastating complications of bowel anastomoses, often requiring additional procedures and/or surgery, and can lead to sepsis, critical illness, and death.

A continuously bonded anastomosis with exact mucosal approximation may improve anastomotic healing by minimizing collagen exposure to bacteria. Exact mucosal approximation may be achieved through a meticulous ‘mucosal exclusion’ hand-sewn suturing technique. While very low leak rates have been reported with this technique, it is time consuming and technically difficult to perform.

Some highly experimental anastomotic techniques have been reported in ex vivo animal models, but the research has not translated to human trials since none have been demonstrated to be superior to existing sutures and staples. Such techniques include bipolar electrocautery tissue fusion and use of surgical adhesives. However, these existing techniques still utilized inverted tissue edges leaving exposed collagen at the anastomotic wound.

Bipolar electrocautery passes electric current across two layers of tissue such that the proteins at the interface of the tissue layers denature, become entangled, and fuse upon cooling. Some known bipolar electrocautery procedures utilize a thermoelectric fusion device where two cut bowel ends are secured around a disk of the fusion device and electrodes are used to perform the fusion. The burst pressure tolerance of bipolar electrocautery anastomoses can be greater than that of hand-sewn anastomoses. However, the fusion device requires an additional enterotomy to use, similar to an end-to-end stapling device.

For adhesive anastomoses, one experimental trial described using a circular stapling device with the staples removed to approximate and compress bowel ends coated with a surgical adhesive such as cyanoacrylate. Similar to other methods, this inverts the bowel edges and an additional enterotomy is required. This experimental trial reported that the cyanoacrylate anastomosis was capable of withstanding pressures greater than those generated by a normal human bowel, but are inferior to stapled anastomoses. Additionally, cyanoacrylate causes an inflammatory reaction when used internally and is only FDA approved for topical use.

Another experimental technique used a biodegradable stent over which a sutured bowel anastomoses was performed. Widened ends of the stent assisted in holding the stent in place while sutures are tied around the outside of each bowel end. Additional sutures are used to approximate the bowel ends where the bowel ends meet at the center of the stent. However, this technique was only demonstrated in vivo, using a pig model, and showed no advantage over traditional techniques.

A compressive anastomotic device, named the ‘Murphey Button’ was first described in the 1800s and does not utilize sutures or staples. The Murphey Button is comprised of two spring-loaded interlocking rings, over which the cut bowel ends are secured. The pressure between the rings creates an initial seal, and over time fuses and cuts the bowel layers. The button then passes through the GI tract and is evacuated. However, the compressive anastomotic devices can cause complications including intestinal obstruction, delayed stricture and requires an inverted, exposed cut bowl for securing the compressive rings.

As discussed above, healing of anastomotic wounds, (e.g., wounds created by surgical repair and/or surgical connection of two opposing segments of the bowel following resection of a portion of the bowel) is impaired by the adherence of gut bacterial to exposed collagen at the wound site. Collagenolytic bacteria may be implicated in some clinically relevant anastomotic leaks. Sutured and stapled anastomoses leave collagen exposed in the anastomotic wound, potentially increasing the risk of bacteria-mediated breakdown. Mucosal approximation by meticulous hand-sewn technique has been shown to improve healing and decrease leak rates; however, this technique is difficult to perform. Several continuous bonding technologies have been investigated, although not in a manner that carefully approximates mucosa.

Therefore, a need exists for anastomotic techniques that reduce leak rate by limiting exposed collagen in the wound.

### SUMMARY

One aspect of the present disclosure is directed to a coupling device for constructing bowel anastomoses between a first tubular segment and a second tubular segment. The coupling device includes a wall having a first end and an opposing second end. The wall defines a bore. The first end is sized and shaped for insertion into the first tubular segment and the second end is sized and shaped for insertion into the second tubular segment. The coupling device is configured to support the first and second tubular segments when the first end is inserted into the first tubular segment and the second end is inserted into the second tubular segment. The wall includes a mucoadhesive polymer configured to adhere to the first tubular segment and the second tubular segment.

Another aspect of the present disclosure is directed to a method of manufacturing a coupling device. The method includes rotating a first ring and a second ring. The first ring and the second ring are spaced apart from each other and supported by a drum. The method includes applying a mucoadhesive polymer solution to the rotating first and second rings supported by the drum such that the polymer solution builds-up between the first and second rings creating a wall defining a bore. The wall is attached to the first and second rings.

Yet another aspect of the present disclosure is directed to a method for constructing anastomoses between a first tubular segment and a second tubular segment. Each of the first and second tubular segments has a mucosal edge and a lumen. The method includes inserting a first ring of a coupling device within a first lumen of the first tubular segment and inserting a second ring of the coupling device within a second lumen of the second tubular segment. The coupling device is configured to support the first and second tubular segments. The method includes adhering the coupling device to the first and second tubular segments for alignment of the mucosal edges of the first and second tubular segments at a mucosal approximation site.

Various refinements exist of the features noted in relation to the above-mentioned aspects of the present disclosure. Further features may also be incorporated in the above-mentioned aspects of the present disclosure as well. These refinements and additional features may exist individually or in any combination. For instance, various features discussed below in relation to any of the illustrated embodiments of the present disclosure may be incorporated into any of the above-described aspects of the present disclosure, alone or in any combination.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1 is a perspective view of an embodiment of a coupling device for anastomosis;

FIG. 2 is a perspective end view of the coupling device shown in FIG. 1;

FIG. 3 is a side view of the coupling device shown in FIG. 1;

FIG. 4 is a perspective view of an anastomoses with two opposing cut bowel segments which are bonded together using an outer application of a flexible albumin based surgical glue;

FIG. 5 is a schematic of a cross-sectional view of the coupling device supporting an anastomoses with two opposing cut bowel segments;

FIG. 6 is a schematic of a system for manufacturing the coupling device shown in FIG. 1;

FIG. 7 shows an anastomosis permeability assay system for experimentally evaluating anastomosis permeability;

FIG. 8 is a bar graph comparing the concentration of extraluminal fluorescein over time for anastomosis formed using different techniques, the extraluminal fluorescein is measured using the anastomosis permeability assay system shown in FIG. 7; and

FIG. 9 is an image of sample experimental bacterial growth resulting from bacteria anastomosis permeability using the anastomosis permeability assay system shown in FIG. 7.

Corresponding reference characters indicate corresponding parts throughout the drawings.

### DETAILED DESCRIPTION

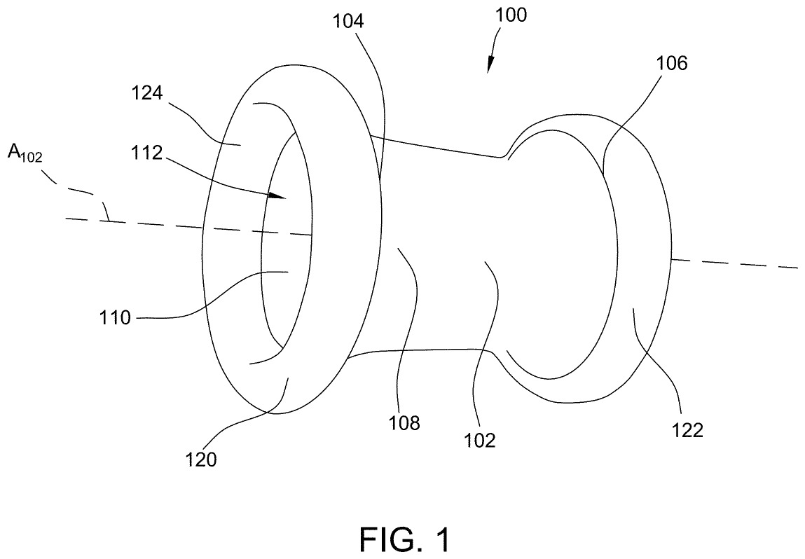

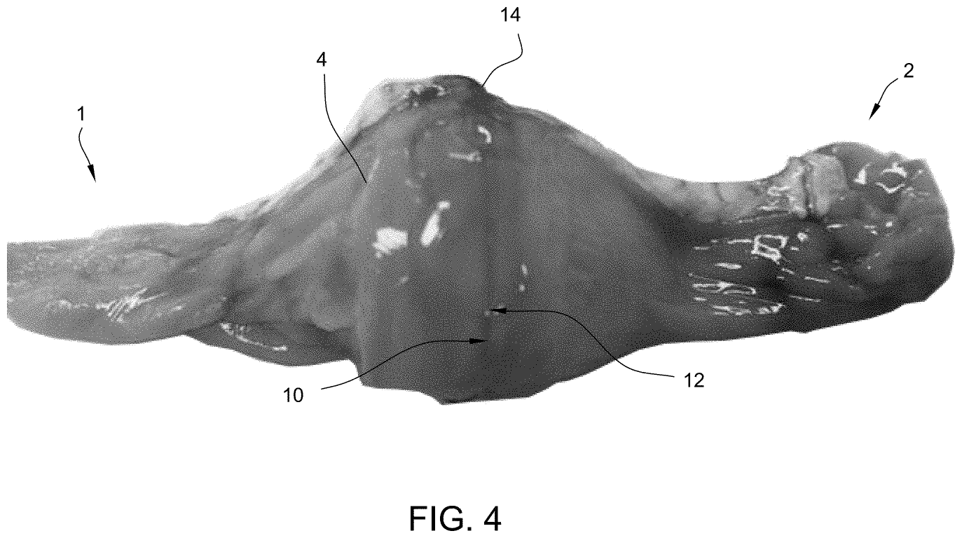

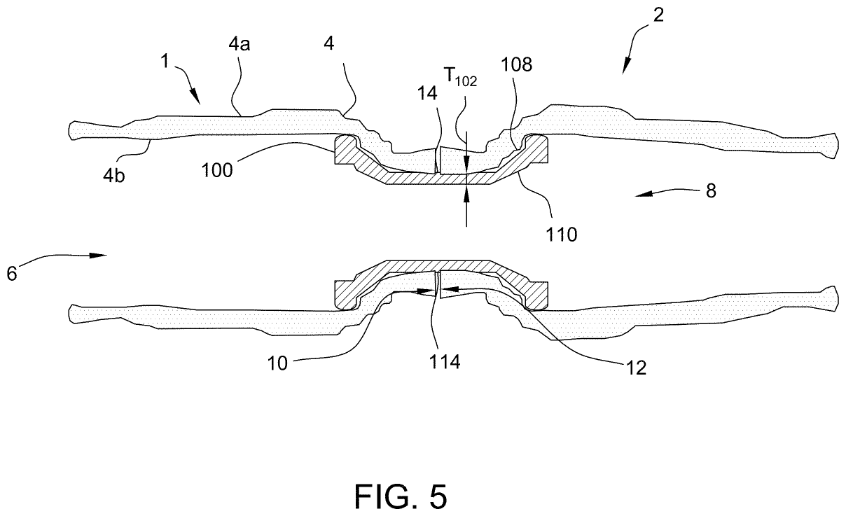

The present disclosure is generally directed towards devices and methods of performing end-to-end intestinal (i.e., bowel) anastomosis between a first tubular segment 1 and an opposing second tubular segment 2 (FIGS. 4 and 5). With reference now to FIG. 1, one suitable embodiment of a coupling device for use during anastomosis is indicated generally by 100.

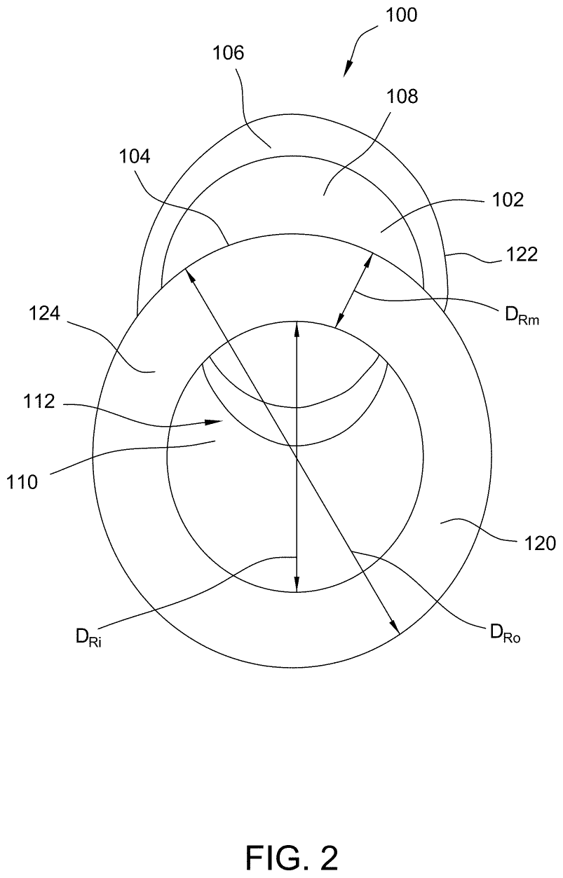

With reference to FIGS. 1-3, the coupling device 100 includes a generally cylindrical wall 102 having a first end 104 and an opposing second end 106. The wall 102 includes a wall axis A₁₀₂ that extends from the first end 104 to the second end 106. The wall 102 includes an outer surface 108 and an opposing inner surface 110. The inner surface 110 defines the boundary of a bore 112 that extends from the first end 104 to the second end 106 along the wall axis A₁₀₂.

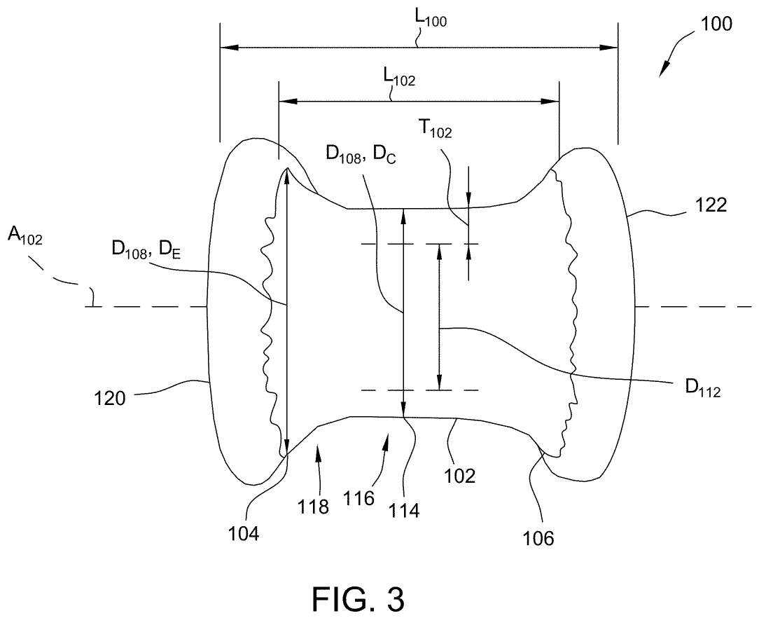

As seen in FIG. 3, the wall 102 includes a thickness, T₁₀₂, between the inner surface 110 and the outer surface 108. The outer surface 108 of the wall 102 defines a wall outer diameter D₁₀₈. The wall thickness T₁₀₂, is generally uniform. The wall thickness T₁₀₂ may be between 0.2 cm and 0.7 cm. In some embodiments, the wall 102 may have a non-uniform thickness.

The wall 102 extends radially about the coupling axis A₁₀₂ such that the bore 112 is generally cylindrical such that a cross-section taken perpendicular to the wall axis A₁₀₂ is circular. The bore 112 has a bore diameter D₁₁₂, which corresponds to a wall inner diameter. Alternatively, the wall 102 may be shaped such that the bore 112 is substantially elliptical in cross-section or any other suitable shape.

The bore diameter D₁₁₂ and the outer diameter D₁₀₈ may be generally constant along a portion of the coupling device 100. For example, the coupling device 100 includes a mid-point 114 halfway between the first end 104 and the second end 106. The bore diameter D₁₁₂ and the outer diameter D₁₀₈ are generally constant in a first region 116 that extends on either side of the mid-point 114. In the illustrated embodiment, the bore diameter D₁₁₂ and the outer diameter D₁₀₈ increase in second regions 118 that extend from the first region 116 to the first end 104 and the second end 106. The second regions 118 are on either side of the first region 116.

The first region 116 may extend a longer distance along the coupling axis A₁₀₂ than the second regions 118. For example, the first region 116 may extend along a majority, i.e., greater than 50%, of the length of the coupling device 100 and the second regions 118 may cumulatively extend along less than 50% of the length of the coupling device 100.

With reference again to FIGS. 1-3, the coupling device 100 includes a first ring 120 coupled to the wall 102 at the first end 104 and second ring 122 attached to the wall 102 at the second end 106. In some suitable embodiments, the first and second rings 120, 122 are formed integrally with the wall 102. The first ring 120 and the second ring 122 have an outer ring diameter D_{Ro} and an inner ring diameter D_{Ri}. For example, the outer ring diameter D_{Ro} may be between 2 centimeters (cm) and 6 cm and the inner ring diameter D_{Ri} may be between 1.6 cm and 4 cm. In the exemplary embodiment seen in FIGS. 1-3, the inner ring diameter D_{Ri} is equal to the bore diameter D₁₁₂ such that the interior of the rings 120, 122 is flush with the inner surface 110 when the first ring 120 is attached to the first end 104 and the second ring 122 is attached to the second end 106.

The first and second rings 120, 122 have a curved surface 124 providing a torus shape (i.e., doughnut shape). The curved surface 124 defines the outer ring diameter D_{Ro}, the inner ring diameter D_{Ri}, and a minor diameter D_{Rm}. The minor diameter D_{Rm} is equal to the difference between the outer ring diameter D_{Ro} and the inner ring diameter D_{Ri}. For example, the minor diameter D_{Rm} may be between 0.2 cm and 1 cm. Alternatively, the first and second rings 120, 122 may be any suitable shape, for example and without limitation, cylindrical, conical, rectangular, triangular, polygonal, and/or ovular in shape.

A central diameter D_{c}, defined by the outer surface 108 of the wall 102, generally at the mid-point 114, is between 1 and 4 cm. An end diameter D_{E} defined by the outer surface 108 of the wall 102, in proximity to the first and second ends 104, 106, is greater than then central diameter Dc and is equal to the ring outer diameter D_{Ro}. The central diameter D_{c} is narrower than the ring outer diameter D_{Ro} and end diameter D_{E} such that the coupling device 100 has an overall “dumbbell-like” shape. The ring outer diameter D_{Ro} is sized such that the first and second rings 120, 122 engage with a mucosal wall 4 which defines the boundary of a lumen and retain the position of the coupling device 100 within the lumen as discussed in detail further herein (FIGS. 4 and 5). The central diameter D_{c} is narrower than the ring outer diameter D_{Ro} to relieve stress on the first and second cut tubular segments 1, 2 in proximity to a mucosal approximation site 14 when the segments 1, 2 are positioned on the coupling device 100.

As illustrated in FIG. 3, the wall 102 has a length L₁₀₂ extending between the first end 104 and the second end 106 along the wall axis A₁₀₂. The length L₁₀₂ may be between 2 and 10 cm (FIG. 3). The coupling device 100 has an overall length L₁₀₀ that includes the length L₁₀₂ of the wall 102 and the minor diameter D_{Rm} of the first and second rings 120, 122. The overall length L₁₀₀ may be between 2.4 cm and 12 cm.

The wall 102 and the first and second rings 120, 122 may be composed of a suitable biocompatible material. As used herein, the term “biocompatible material” means that the material does not have toxic or injurious effects on biological tissues and is suitable to be in contact with living systems without producing an adverse effect. The wall 102, and potentially the first and second rings 120, 122, are composed of a biocompatible mucoadhesive polymer, as discussed in further detail herein.

In suitable embodiments, the coupling device 100 may have any suitable shape. For example and without limitation, the wall 102 may include a generally cylindrical shape wherein the bore 112 defined by the wall 102 has a generally constant diameter along the entire length L₁₀₂ of the wall 102.

The dimensions of the coupling device 100, including the wall length L₁₀₂, the overall length L₁₀₀, the bore diameter D₁₁₀, the outer diameter D₁₀₅, and the dimensions of the first and second ring 120, 122, etc. each may be scaled and/or altered to be suitable for various sizes and cases of anastomosis.

In reference now to FIGS. 4 and 5, the coupling device 100 is used for performing an end-to-end anastomosis between the first and second tubular segments 1, 2. For example, the tubular segments 1, 2 may be segments of a bowel that have been cut or otherwise separated from each other or other portions of the bowel. Each of the first and second tubular segments 1, 2 includes a mucosal wall 4 that defines the boundary of a lumen (FIG. 5), i.e., a first lumen 6 and a second lumen 8, respectively. The mucosal wall 4 of the first tubular segment 1 includes a first end surface 10 and the second tubular segment 2 includes a second end surface 12 (e.g., the first and second end surface 10, 12, are a cut end of the first and second tubular segments 1, 2 and/or an edge of the mucosal wall 4 edge). The mucosal wall 4 also includes a mucosal outer surface 4a and a mucosal inner surface 4b, which opposes the mucosal outer surface 4a.

During anastomosis, the coupling device 100 is placed within the first and second lumen 6, 8. More specifically, the first end 104 and the first ring 120 are inserted into the first lumen 6 and the second end 106 and the second ring 122 are inserted into the second lumen 8. The coupling device 100 is inserted into the first and second lumen 6, 8 and the walls 4 are positioned over the coupling device 100 such that the first and second end surfaces 10, 12 circumferentially align and meet at a mucosal approximation site 14 (FIGS. 4 and 5), generally at the mid-point 114 of the coupling device 100. Accordingly, approximately half of the coupling device 100 is disposed within the first lumen 6 and the other half the coupling device 100 is disposed within the second lumen 8. The first and second rings 120, 122 are sized such that the ring outer diameter Dᵣₒ is greater than the size of the first and second lumen 6, 8 causing the first and second rings 120, 122 to engage with (e.g., press outward against) the mucosal wall 4. FIG. 4 depicts a completed anastomosis after the biodegradable coupling has dissolved away such that only the surgical glue joins the first and second tubular segments 1, 2.

The wall 102 of the coupling device 100 may be comprised of a mucoadhesive polymer. The mucoadhesive polymer may adhere and/or substantially couple to the mucosal wall 4 of the first and second the tubular segments 1, 2. Specifically, the mucosal inner surface 4b adheres to the outer surface 108 of the wall 102. Mucoadhesive polymers adhere to mucosal surfaces through a variety of interface interactions including electrostatic forces and covalent bonds. Adherence of the mucosal wall 4 to the wall 102 enables the coupling device 100 to retain the position of the first and second end surfaces 10, 12 at the mucosal approximation site 14. The adherence between the wall 102 and the mucosal wall 4 is sufficient such that the coupling device 100 is resistant to migration due to peristalsis.

The mucosal walls 4 of the first and second tubular segments 1, 2 temporarily adhere to the coupling device 100 such that the coupling device 100 supports the first and second tubular segments 1, 2 and retains the alignment between the first and second end surfaces 10, 12 for mucosal approximation during anastomosis. The orientation of the mucosal walls 4 using the coupling device 100 is unique compared to conventional methods of performing anastomoses because mucosal approximation using the coupling device 100 does not require inverting and/or everting the mucosal wall 4. Embodiments of the present disclosure allow the first and second end surfaces 10, 12 to be generally perfectly approximated and circumferentially aligned, also referred to herein as exact mucosal approximation, promoting more rapid healing compared to other known anastomosis methods that require inverting or everting the mucosal wall 4.

In the illustrated embodiment, for example, the biocompatible mucoadhesive polymer is a chitosan polymer. Chitosan is a mucoadhesive polymer derived from chitin treated with an alkaline substance; commercially available products made from chitosan are generally manufactured from shrimp shells. Given its availability, biocompatibility, and intrinsic antimicrobial properties, several varieties of solvent casting and electrospinning techniques have been described to fabricate clinically useful bioactive materials from chitosan polymer solutions as described in further detail herein. In some embodiments, the biocompatible mucoadhesive polymer may include at least one of Alginate, Hyaluronic Acid, Pullulan, Carbopol, Poly Lactic-co-Glycolic Acid (PLGA), Polylactic Acid (PLA), Polyacrylates, Polyethylene Glycol, and polyethylene Oxide. Alternatively, the biocompatible mucoadhesive polymer may include any suitable material that is biocompatible and includes properties that enable adherence to mucosal surfaces.

The coupling device 100 is at least partially biodegradable such that at least a portion of the coupling device 100 breaks down after placement within the body. For example, the wall 102 may be made from a biodegradable drug eluting material, which locally delivers pharmacologic and/or bioactive agents to the first and second tubular segments 1, 2. For example, the wall 102 is composed of a mucoadhesive polymer, e.g., chitosan, which is biodegradable and has been dosed with bioactive agents (i.e., bioactive agents are embedded in the polymer such that they are generally evenly dispersed throughout the polymer). After placement of the coupling device 100 within the first and second lumen 6, 8, the wall 102 biodegrades (i.e., breaks down and dissolves within the body) releasing the bioactive agents embedded therein, into the nearby mucosal wall 4 and the first and second lumen 6, 8, in proximity to the mucosal approximation site 14. The wall 102 may completely biodegrade within 2-3 hours after implantation into the body, leaving the first and second rings 120, 122 which may then be subsequently evacuated. In alternative embodiments, the wall 102 is comprised of any suitable material that enables the coupling device 100 to function as described herein.

The first and second rings 120, 122 are composed of an elastic material (i.e., flexible), such as a biocompatible rubber or a medical grade rubber. In some embodiments, the first and second rings 120, 122 may be composed of silicone. Additionally or alternatively, the first and second rings 120, 122 may be composed of the mucoadhesive polymer and/or the first and second rings 120, 122 may be biodegradable and as such, the first and second ring 120, 122 do not require subsequent evacuation.

In some embodiments, locally delivered pharmacologic (i.e., bioactive agents) such as polyphosphates, erythropoietin, and doxycycline inhibit bacterial action and improve healing of the anastomotic wound. Polyphosphate, delivered both orally or by enema, suppresses collagenolysis and prevents of anastomotic breakdown. Additionally, tranexamic acid may play a role in plasmin modulation and inhibit bacteria-mediated breakdown.

Given the involvement of matrix metallopeptidases (MMPs) with collagen remodeling, a MMP inhibitor such as doxycycline may also be used for the bioactive agent. For example, doxycycline may be placed within a porcine colorectal anastomosis to locally decrease MMP activity. The doxycycline does not negatively affect strength or healing of the new connection as compared to other anastomoses. Additionally, erythropoietin and granulocyte macrophage colony stimulating factors (two types of growth factors) may be used to increase angiogenesis and recruitment of fibroblasts to the wound site, thereby resulting in enhanced healing demonstrated by superior tensile strength of the anastomosis.

The bioactive agents may include any suitable bioactive agents capable of inhibiting bacteria-mediated anastomotic breakdown (i.e., anti-bacterial properties) and thereby improving healing of anastomoses. In some embodiments, bioactive agents include for erythropoietin and/or doxycycline. Alternatively and/or additionally, the bioactive agents may include, for example and without limitation, polyphosphate compounds and/or tranexamic acid or any combination of the aforementioned listed bioactive agents. Alternatively and/or additionally, the bioactive agents may include any suitable agents, which are enabled to improve anastomotic healing.

As the coupling device 100 supports and maintains the alignment of the tubular segments 1, 2, continuous bonding techniques may be used to bond together the first and second end surfaces 10, 12 of the first and second tubular segments 1, 2. Continuously bonding anastomosis may be accomplished using, for example and without limitation, surgical adhesives, bipolar electrocautery, and/or compression devices. Continuous bonded anastomosis may include inverting or everting the mucosal walls, which exposes collagen to bacteria, inhibiting healing of the anastomotic wound, as discussed previously. More advantageously, continuously bonding anastomosis with exact mucosal approximation, wherein the first and second end surfaces 10, 12 are bonded together to align circumferentially, without inverting or everting the mucosal wall 4, minimizes collagen exposure and reduces bacteria breakdown.

FIG. 4 shows a continuously bonded anastomosis using an albumin based surgical glue. Other continuous bonding anastomosis may be used with the embodiments described herein without departing from some aspects of the disclosure. For example, the interrupted serosubmucosal anastomosis is a hand-sewn technique that excludes the mucosa, passing interrupted sutures only through the outer bowel layers. This meticulous technique in theory affords better approximation of and blood supply to the mucosal layer, allowing rapid healing and sealing of exposed collagen. Once the bowel is immobilized by the coupling device 100 (i.e., through adherences of the coupling device to the first and second tubular structures 1, 2), sutures may be placed to hold and/or join the tubular segments 1, 2 together to allow for healing after at least a portion of the coupling device 100 has dissolved. The tubular segments 1, 2 may be joined together while the tubular segments 1, 2, in proximity to the mucosal approximation site 14, are immobilized by the coupling device 100. For example, surgical glues, sutures, staples, clips, bipolar electrocautery, compression devices, or adhesive wraps individually or in combination, may be used to attach and join the tubular segments 1, 2 together, while the wound heals.

Surgical glues may be used to continuously bond the first and second tubular segments 1, 2 to achieve exact mucosal approximation, while the coupling device 100 retains the position of the first and second surfaces 10, 12. Surgical glues include biocompatible adhesives and/or sealants, for example and without limitation, cyanoacrylate, albumin, fibrin, and polyethylene-glycol based surgical glues. In the illustrated embodiment, a two-part albumin based surgical adhesive, which can be used for reinforcement of cardiovascular anastomoses, is used to continuously bond the first and second tubular segments 1, 2. FIG. 4 illustrates an anastomosis bonded using an external application of a flexible albumin-based surgical adhesive. The two-part albumin cures in wet conditions (i.e., within the body) within minutes, resulting in a durable and flexible material, with properties similar to silicone. In alternative embodiments, continuous bonding techniques for mucosal approximation may utilize other types of surgical adhesives, which enable the coupling device 100 to function as described herein.

Biocompatible adhesives have been developed that are candidates for purely adhesive bowel anastomoses. Some of these glues utilize laser energy to catalyze a chemical bond between tissue and internal or external scaffold, which has been respectively demonstrated in blood vessel and nerve animal models. Commercially available surgical adhesives and sealants fall into four categories: cyanoacrylate, albumin, fibrin, and polyethylene-glycol based glues. Anastomoses may be performed with inverted tubular segments with adhesion between external serosal surfaces. Comparing the tensile, shear, and peel strength among the various adhesives and sealants, the cyanoacrylates performed the best, followed by albumin based glues.

Adhesives and sealants differ in that adhesives are able to withstand greater tensile forces. Surgical sealants composed of human fibrin and/or thrombin have been used to seal the exterior of traditional hand-sewn and stapled colorectal anastomoses in human clinical trials. While sealing may seem like an intuitive solution to anastomotic leaks, external sealants fail to address bacterial exposure to collagen. In contrast to conventional systems and methods, the coupling device 100 (shown in FIG. 1) may be used to support the tubular segments 1, 2 which enables an externally continuously bonded anastomosis with exact mucosal approximation reducing collagen exposure and improving healing. In addition, the coupling device 100 locally delivers bioactive agents, impeding bacterial action while the mucosa heals.

In some embodiments, the continuous bonding techniques include the application of an outer adhesive wrap (not shown) to support the continuous bond between the first and second tubular segments 1, 2. The outer adhesive wrap increases the surface area supporting the mucosal approximation site 14, distributes peristaltic forces over a surface area of the adhesive wrap, and reduces shear forces experienced at the mucosal approximation site 14. The outer adhesive wrap acts to support the surgical glue at the mucosal approximation site 14 (shown in FIG. 4). The outer adhesive wrap may include a woven oxidized cellulose and/or polyglactin mesh. In some embodiments, the outer adhesive wrap includes an adhesive barrier such as sodium hyaluronate/carboxymethylcellulose, which is conventionally used in clinical settings.

Tensile strength testing of anastomoses indicates that continuous bonded techniques using surgical glues may be about half as strong as sutured and/or hand-sewn anastomoses. However, sutures and staples can withstand forces far greater than the bowel can generate, so half-strength may be adequate in vivo. In some samples, the continuously bonded connections failed due to fracturing of the surgical glue between the first and second tubular segments 1, 2 (i.e., as opposed to fracturing at the interface between the surgical glue and the mucosal wall). The fracture in the surgical glue indicates that there is relatively strong adhesion between the mucosal wall 4 and the surgical glue.

In described examples, the continuous bonding technique using the surgical glue is supported by the coupling device 100 which retains the position of the first and second tubular segments 1, 2 as the surgical glue dries and/or cures. Additionally or alternatively, the first and second tubular segments 1, 2 may be attached together using mechanical fasteners, e.g., staples or sutures. The coupling device 100 acts to stabilize the anastomosis while the first and second tubular segments 1, 2 are joined together continuously, using surgical glue, or otherwise are structurally connected. In some embodiments, the surgical glue and/or adhesive is be applied external, to the mucosal outer surface 4a, such that the surgical glue spans across the mucosal approximation site 14. In other embodiments, bipolar or compressive techniques may be used to connect the first and second tubular segments 1, 2 while the anastomosis is supported by the coupling device 100. Additionally and/or alternatively, joining the first and second tubular segments 1, 2 together may include using at least one or more of the following: surgical glues, sutures, staples, clips, bipolar electrocautery, compression devices, and adhesive wraps.

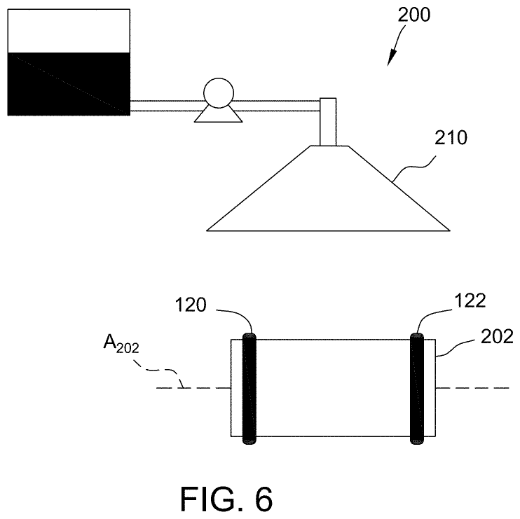

In reference to FIG. 6, the coupling device 100 may be manufactured using a system 200 in a rotational solvent-casting method. The system 200 includes a drum 202. The drum 202 is generally cylindrical in shape. The drum 202 includes a longitudinal drum axis A₂₀₂ about which the drum 202 rotates. For example, the drum 202 may be connected to a motor (not shown) which rotates the drum 202 at any suitable RPM. The drum 202 has a diameter such that the drum 202 may support the first and second rings 120, 122, e.g., the first and second rings 120, 122 may be press fit over the drum 202. In some embodiments, the first and second rings 120, 122 may be coupled to the drum 202 or otherwise attached to the drum 202. The drum axis A₂₀₂ is directed generally through a center of the first and second rings 120, 122 when they are arranged over the drum 202. A solution, such as liquid mucoadhesive polymer, (e.g., liquid concentrated chitosan polymer) may be applied to the drum 202 as it is rotated. For example, an applicator 210 may be used to apply solution to the drum 202. In some embodiments, a nozzle may be used to direct a stream or spray of the solution towards the drum 202 and the first and second rings 120, 122.

A casting method, using system 200, includes placing the first and second rings 120, 120, spaced apart by a distance, over the drum 202. The method may include coupling the first and second rings 120, 122 to the drum 202. The solution is then evenly applied onto at least a portion of the rotating drum 202. The solution may also be evenly applied onto at least a portion of the first and second rings 120, 120. In some embodiments, the solution is sprayed onto the rotating drum. The solution builds-up between the first and second rings 120, 122, as the solution is coated over the rotating drum 202.

The solution that has built-up over the drum 202 may be dried using a dryer (e.g., a heater and/or fan) and/or air-dried, forming the wall 102 of the coupling device 100 between the first and second rings 120, 122. The wall 102 may contract during drying, causing the central diameter D_{c} to be narrower than the ring outer diameter D_{Ro} and end diameter D_{E} such that the coupling device 100 has an overall “dumbbell-like” shape. The solution may be doped with bioactive agents, such that the bioactive agents will be embedded within the wall 102. In other embodiments, bioactive agents may be applied to the outer surface 108 of the wall 102.

In reference to FIGS. 1-4, a method for constructing an anastomosis between the first and second tubular segments 1, 2, using the coupling device 100 includes inserting the first end 104 and the first ring 120 into the first lumen 6 and inserting the second end 106 and the second ring 122 into the second lumen 8, such that the first and second end surfaces 10, 12 circumferentially align at generally the mid-point 114 of the coupling device 100. The method includes adhering the coupling device 100 to the first and second tubular segments 1, 2 such that the coupling device 100 maintains the alignment of the first and second end surfaces 10, 12 at the mucosal approximation site 14. The method further includes bonding the first and second end surfaces 10, 12 together using the continuous bonding techniques, such as applying surgical glue to the first and second end surfaces 10, 12 thereby gluing the end surfaces 10, 12 together. Biodegradation of the wall 102 or at least a portion of the coupling device 100 releases bioactive agents to the surrounding mucosal wall 4, leaving the remaining first and second rings 120, 122 disposed within the first and second lumen 6, 8. The method may also include removing the first and second rings 120, 120 using non-invasive or minimally invasive surgical procedures or other means. In embodiments in which the first and second rings 120, 122 are biodegradable, the first and second rings 120, 122 do not need to be removed or evacuated.

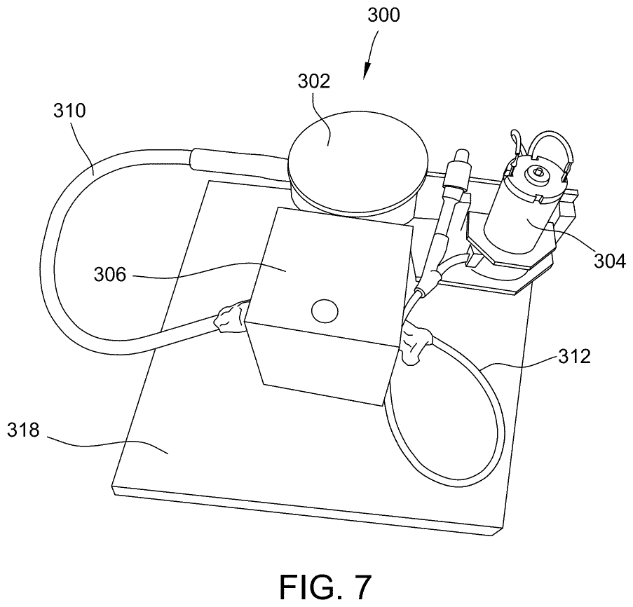

A benefit of continuous bonding techniques using surgical glues as compared to other methods, e.g., using sutures and/or staples, is that using the surgical glue to continuously bonds the first and second end surfaces 10, 12 limits bacterial migration across the collagen rich anastomotic wound. In reference to FIG. 7, a low-pressure anastomosis permeability assay system 300 may be used to measure anastomotic permeability (also referred to herein as leaking and/or leak rate). Anastomotic permeability is a measure of the passage of a material (e.g., bacteria) across the anastomotic wound, for example, from an extraluminal space (i.e., a space outside the mucosal wall) to an intraluminal space (i.e., within the first and second lumen 6, 8 of the first and second tubular segments 1, 2). Additionally or alternatively, anastomotic permeability is a measure of the passage of materials from the intraluminal space to the extraluminal space.

In reference again to FIG. 7, the anastomosis permeability assay system 300 includes a reservoir 302 and a pump 304 fluidically connected to the reservoir 302. The system 300 includes a testing chamber 306 that is fluidically connected to the pump 304 and the reservoir 302. The testing chamber 306 is sized and shaped to receive a specimen anastomosed tubular segment for evaluating anastomotic permeability. The reservoir 302 stores a first fluid containing a marker. The first fluid, representative of the intraluminal fluid, is pumped into the intraluminal space within the specimen anastomosed tubular segment via a first catheter 310. A second catheter 312 drains the fluid back into reservoir 302. The catheters 310, 312 may be any suitable catheter. For example, in some embodiments, the catheters 310, 312 are Foley type catheters.

The testing chamber 306 contains a second fluid, representative of the extraluminal fluid, which surrounds the outside of the specimen anastomosed tubular segment. In some embodiments, a first and second Foley balloon (not shown) may keep the intraluminal fluid isolated from the extraluminal fluid within the test chamber.

In this illustrated embodiment, the marker is fluorescein, which was selected for its stability and straightforward spectrophotometric quantification. In alternative embodiments, additional and/or alternative suitable markers may be used evaluate anastomotic permeability. The extraluminal fluid is iteratively sampled over a period of time and tested for presence of the marker to quantify leak rate.

In some embodiments, the anastomosis permeability assay system 300 may be placed on a hotplate-stirrer 318 to continuously agitate the extraluminal fluid to distribute any leaked marker throughout the extraluminal fluid, ensuring even sampling. The pump 304 produces an intraluminal pressure of approximately 2 mmHg, to simulate the environment of a postoperative ileus. In other embodiments, the pump 304 produces any suitable low intraluminal pressure that enables the anastomosis permeability assay system 300 to function as described herein.

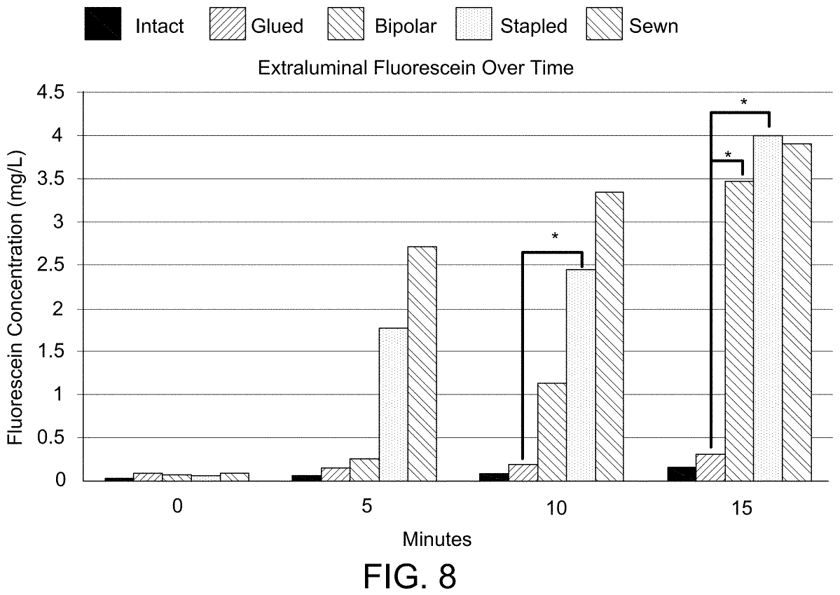

In reference to FIG. 8, the anastomosis permeability assay system 300 (FIG. 7) was used to evaluate anastomoses permeability for five different specimens of anastomosed tubular segments. The five specimens included a completely intact tubular segment, a continuously connected anastomoses using surgical glue (FIG. 4), a bipolar attached anastomoses, a stapled anastomoses, and a hand-sewn anastomoses. Four to six trials were completed for each of the five different anastomosed bowel specimens. All trials were performed by a single researcher, except for the hand-sewn anastomoses for which was performed by an additional researcher to reduce bias.

FIG. 8 is a bar graph of the results of the anastomoses permeability for the five anastomoses specimens evaluated at time increments of 0 min, 5 min, 10 min, and 15 min. Anastomoses permeability was evaluated by sampling extraluminal fluid and determining the concentration of fluorescein marker contained within the sample, at each of the time increments. Minimal fluorescein marker concentration, below 0.25 mg/L, was detected for both the complete intact tubular segment and the continuously connected anastomoses at a 15 min time increment. A fluorescein marker concentration over 3 mg/L was detected in each of the bipolar attached, stapled, and hand-sewn anastomoses, sampled at the 15 min time increment. There was not a significant difference between the continuously connected anastomosis and hand-sewn tubular segments, given the wide variance in hand-sewn leak rates.

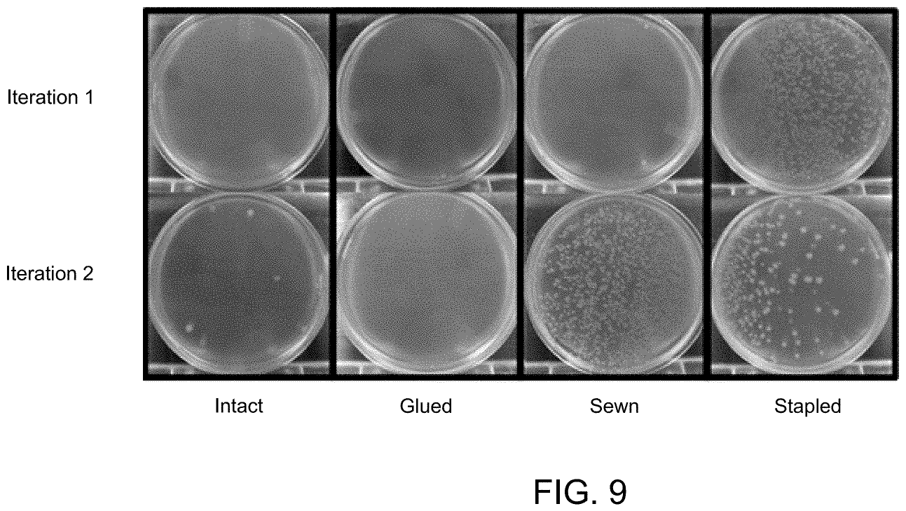

In reference to FIG. 9, the anastomosis permeability assay system 300 (FIG. 7) was used to evaluate anastomoses permeability of bacteria for four different specimens of anastomosed tubular segments for two iterations. The four specimens evaluated included: a completely intact tubular segment, a continuously connected anastomoses using surgical glue (FIG. 4), a stapled anastomoses, and a hand-sewn anastomoses. The two iterations of the hand-sewn anastomosis were performed by two different researchers. Bacteria cells are typically much larger than the fluorescein molecules that were used to determine anastomoses permeability in the previously described experiment (FIG. 8). Bacteria were introduced to the extraluminal fluid and the intraluminal solution was sampled to detect the presence of bacteria that entered into intraluminal space. Samples of intraluminal fluid were plated on Petri dishes and incubated for 36 hours. FIG. 9 shows colonies for each of the evaluated specimens for the two iterations. Colonies represent individual bacteria cells that have passed across the anastomotic wound from the extraluminal space to the intraluminal space. The colonies in the second intact trial likely represent incomplete decontamination, as the second intact trial was performed using the system 300 in the trial after the first stapled trial was evaluated.

Potential testing, including animal and human trials, may be used to improve outcomes of anastomosis using the coupling device 100 and the methods and systems described above without departing significantly from the claimed embodiments. Potential future work includes the following benchtop experimentation. This work will refine the embodiments described above and may be necessary for potential FDA approval of the systems and methods of the embodiments described above.

The methods and systems described herein may be used for many applications. For example, the described apparatus and methods may be used for high-risk clinical scenarios such as penetrating trauma or sepsis, where a bacteria-resistant anastomosis may make it possible to avoid fecal diversion. In addition, the described apparatus and methods may be used for end-to-side and colorectal anastomoses.

Compared to conventional methods and systems of performing end-to-end anastomosis between opposing tubular segments, embodiments of the present disclosure have several advantages. Embodiments of the coupling device is composed of a biocompatible, biodegradable, mucoadhesive drug eluting material, which adheres to the tubular segments and retains the position of a first and second end surfaces. The coupling device is substantially dumbbell-like in shape to facilitate retention of the coupling device within the lumen of the tubular segments and relieve stress near or at the mucosal approximation site. The coupling device biodegrades to release bioactive agents which inhibit bacterial growth, improving healing of the anastomosis. In addition, the coupling device supports and adheres to the mucosal walls to retain the position of the end surfaces. A surgical glue may be applied to the bond the two end surfaces together for complete bonding of the exact mucosal approximation and to prevent migration of bacteria across the anastomotic wound.

As used herein, the terms “about,” “substantially,” “essentially,” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a,” “an,” “the,” and “said” are intended to mean that there are one or more of the elements. The terms “comprising,” “including,” “containing,” and “having” are intended to be inclusive and mean that there may be additional elements other than the listed elements. The use of terms indicating a particular orientation (e.g., “top,” “bottom,” “side,” etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A coupling device for constructing bowel anastomoses between a first tubular segment and a second tubular segment, the coupling device comprises a wall having a first end and an opposing second end, the wall defining a bore, the first end sized and shaped for insertion into the first tubular segment, the second end sized and shaped for insertion into the second tubular segment, wherein the coupling device is configured to support the first and second tubular segments when the first end is inserted into the first tubular segment and the second end is inserted into the second tubular segment, the wall comprising a mucoadhesive polymer configured to adhere to the first tubular segment and the second tubular segment.

2. The device in accordance with claim 1, wherein the mucoadhesive polymer is at least one of following Chitosan, Alginate, Hyaluronic Acid, Pullulan, Carbopol, PLGA, PLA, Polyacrylates, Polyethylene Glycol, and polyethylene Oxide.

3. The device in accordance with claim 1, wherein the coupling device includes at least one bioactive agent and is at least partially biodegradable, the coupling device releases the bioactive agent to the first and second tubular segments as the coupling device biodegrades.

4. The device in accordance with claim 1, wherein the coupling device has a wall outer diameter including a center diameter near a mid-point and an end diameter near the first and second ends, the center diameter being smaller than the end diameters.

5. The device in accordance with claim 1, wherein the coupling device has a length extending between the first end and second end between 3 cm and 10 cm.

6. The device in accordance with claim 1, wherein the coupling device further comprises a first ring coupled to the wall at the first end and a second ring coupled to the wall at the second end.

7. The device in accordance with claim 6, wherein the first and second rings are each in the shape of a torus having a minor diameter and a ring outer diameter.

8. The device in accordance with claim 6, wherein the first ring and second ring each have an outer diameter between 2 and 6 cm.

9. The device in accordance with claim 6, wherein the first and second rings are comprised of an elastic material.

10. The device in accordance with claim 6, wherein the wall has a uniform thickness.

11. The device in accordance with claim 6, wherein the wall has a thickness between 0.2 cm and 0.7 cm.

12. The device in accordance with claim 6, wherein the first ring and second ring are at least partially biodegradable.

13. A method of manufacturing a coupling device, wherein the method comprises:
rotating a first ring and a second ring, wherein the first ring and the second ring are spaced apart from each other and supported by a drum; and
applying a mucoadhesive polymer solution to the rotating first and second rings supported by the drum such that the polymer solution builds-up between the first and second rings creating a wall defining a bore, wherein the wall is attached to the first and second rings.

14. The method in accordance with claim 13, wherein applying the polymer solution to the rotating first and second rings comprises applying a mucoadhesive polymer solution including chitosan.

15. The method in accordance with claim 13 further comprising positioning the first and second rings on the drum a distance apart such that the wall has a length between 3 cm and 5 cm.

16. The method in accordance with claim 13 further comprising positioning the first ring and the second ring on a first portion of a drum, the first portion of the drum having a first diameter.

17. A method for constructing anastomoses between a first tubular segment and a second tubular segment, each of the first and second tubular segments having a mucosal edge and a lumen, wherein the method comprises:
inserting a first ring of a coupling device within a first lumen of the first tubular segment;
inserting a second ring of the coupling device within a second lumen of the second tubular segment, wherein the coupling device is configured to support the first and second tubular segments; and
adhering the coupling device to the first and second tubular segments for alignment of the mucosal edges of the first and second tubular segments at a mucosal approximation site.

18. The method in accordance with claim 17 further comprising delivering bioactive agents by biodegrading a portion of the coupling device releasing the bioactive agents stored within the coupling device.

19. The method in accordance with claim 17 further comprising joining the first and second tubular segments together.

20. The method in accordance with claim 19, further comprising joining the first and second tubular segments together using at least one of the following surgical glues, sutures, staples, clips, bipolar electrocautery, compression devices, and adhesive wraps, individually or in combination.
